(19) United States
(12) Patent Application Publication
(10) Pub. No.: US 2026/0151248 A1
(43) Pub. Date: Jun. 4, 2026

(54) ADJUSTABLE ORTHOSIS JOINT FOR THE CONTROLLED MOVEMENT AND/OR FIXATION OF A HAND, AND ORTHOSIS COMPRISING SUCH AN ORTHOSIS JOINT

(71) Applicant: KIMEBO UG (HAFTUNGSBESCHRÄNKT), München (DE)

(72) Inventors: Uwe KLIMMER, München (DE); Klaus GANSER, Gilching (DE); Norbert DIEBL, Tutzing / Traubing (DE)

(21) Appl. No.: 18/869,420

(22) PCT Filed: May 19, 2023
(86) PCT No.: PCT/DE2023/100370

§ 371 Date: Nov. 26, 2024

(30) Foreign Application Priority Data
Jun. 2, 2022 (DE) 10 2022 114 050.4
Sep. 1, 2022 (DE) 10 2022 122 114.8

(51) Int. Cl. A61F 5/01

(52) U.S. Cl. CPC A61F 5/013; A61F 2005/0167

(57) ABSTRACT

The present invention relates to an adjustable orthosis joint (2) for the controlled movement and/or fixation of a hand, and to an orthosis (1) comprising such an orthosis joint (2). The orthosis joint (2) according to the invention is characterized by a movement module (21) which comprises at least one base element (213) and at least one control element (212) having a top side (2126) and a bottom side (2127) that faces the base element (213) and is opposite the top side (2126), wherein a rotary cylinder (2121), by way of which the control element (212) is mounted rotatably relative to the base element (213) with respect to a deviation axis (DA), is arranged on the bottom side (2127), wherein the control element (212) is designed such that its rotational movement can be restricted in a controlled manner, and wherein the control element (212) is designed to restrict, in a controlled manner, the range of movement of a pivot lever (22) which is operatively connected to the movement module (21). It can be adjusted by the patient themselves without tools, and is of such compact construction that its use in everyday life is not perceived as disruptive.

The invention relates to an adjustable orthosis joint for the controlled movement and/or fixation of a patient's hand, at least comprising a movement module for controlling an extension and/or flexion movement and for controlling and executing an ulnar and/or radial deviation of the hand; and a pivot lever which is operatively connected to the movement module, for carrying out the extension and/or flexion movement of the hand; and to an orthosis for controlled movement and/or fixation of a hand or a hand and a forearm of a patient with such an orthosis joint.

During the rehabilitation process after a hand injury, the affected hand or forearm of the patient must in many cases be immobilized for a certain period of time. The immobilization usually takes place with hand/forearm orthoses which fix the hand and forearm in a certain position and after the fixation no longer allow any movement of said limbs. However, joints of the hand and/or forearm which are not affected at all by the injury are also inevitably immobilized. Depending on the duration of wearing such an orthosis, this can disadvantageously lead to so-called secondary stiffening of the joints not affected by the injury, the additional mobilization of which after the end of the healing process causes unnecessary costs due to an extended rehabilitation time and thus longer work down times of the patient. In the case of complex injuries to the hand or forearm, it may additionally be desirable to move individual joints that are more advanced in their healing process in a controlled manner and at the same time to continue to fix even more severely damaged joints. In this context, a “controlled movement” is to be understood in particular as a movement that is restricted in its angle of movement. In physiotherapy and orthopedics, for this purpose, orthoses are often used, among other things, which receive limbs and enable the movement of a certain joint in a predetermined angle range, with the result that the patient can carry out the movement independently and safely. The angle range can then be gradually extended depending on the patient's state of training and/or health in order to finally regain the original range of movement.

From the state of the art, such “range-of-movement” (ROM) devices are known in particular for knee orthoses and for the bending and stretching movement of the knee. EP 0 546 331 A1 and EP 0 832 624 A2 disclose, for example, orthosis joints with an adjustable range of movement in relation to a movement in a plane, which can preferably be used in a knee orthosis. U.S. Pat. No. 7,081,102 B1 has also disclosed a carpal tunnel support with a joint, which allows a restricted, radial and/or ulnar deviation of the hand of the user.

The human wrist, however, is characterized by a more complex, “multidimensional” range of movement, which can be described by a combination of an extension/flexion movement (bending and stretching movement in a plane perpendicular to the palm of the hand) and an ulnar or radial deviation (wiping movement in the palm of the hand). The orthosis joints and ROM devices known from the prior art, which can be adjusted in their range of movement, do not allow the control of such a multidimensional movement or require several, separate joints. WO 2021/093 919 A2, US 2021/014 56 20 A1 and CN 210 843 695 U are cited by way of example in this regard. In this context, reference should also be made to DE 10 2019 130 404 A1 of the applicant, which describes an orthosis for controlled movement and/or fixation of a forearm and/or a hand, which takes the described multidimensional movement into account, but it is disadvantageously of comparatively structurally projecting configuration and practically prevents everyday use of the orthosis by users.

Although orthosis joints of the prior art, as known for example from WO 03/070129A1, enable the control of a single degree of freedom of movement in a more compact design, they are unable to reproduce the described, multi-dimensional movement of the wrist.

To solve this problem, an adjustable orthosis joint is already provided by DE 10 2021 121 819.5 of the applicant, which technically takes into account the complex combination of an extension/flexion movement and ulnar or radial deviation. The orthosis joint described therein is of particularly stable design and advantageously enables use during comparatively difficult professional and everyday activities. However, this robustness also entails a certain type of construction which can firstly be adjusted to the individual needs of individual patients only with the help of tools and which certain groups of people, who only have to perform relatively light and moderate work, find too large and thus disturbing when used for a long time.

Proceeding herefrom, the present invention is based on the object of providing an alternative orthosis joint, in particular for a hand orthosis, which improves the existing state of the art in such a way that it allows control of both an extension/flexion movement and ulnar and/or radial deviation of a patient's hand, while being designed to be so user-friendly that it can be adjusted by the patient themselves to the changing individual needs during the progressing rehabilitation without the use of special tools and at the same time is so compact that its use is perceived by the patient in everyday life as pleasant or not disturbing.

This object is achieved by an adjustable orthosis joint having the features of independent claim 1 and by an orthosis having the features of further independent claim 14.

In comparison with orthosis joints of the prior art, the orthosis joint according to the invention is characterized in that the movement module comprises at least one base element and at least one control element with a top side and a bottom side facing the base element and lying opposite the top side,
wherein a rotary cylinder, by way of which the control element is mounted rotatably relative to the base element with respect to a deviation axis, is arranged on the bottom side,
wherein the control element is designed such that its rotational movement around the deviation axis can be restricted in a controlled manner,
and wherein the control element is designed to restrict the range of movement of the pivot lever in a controlled manner.

The adjustable orthosis joint according to the invention makes it advantageously possible to control the angles of the extension and flexion as well as the ulnar and radial deviation of the hand depending on the patient's state of health and need for therapy. The angle of the extension or flexion can be advantageously either fixed at 180°

(|W_{E/F}|=0°; extended hand) or released in a controlled manner in a range of movement of 180°±|W_{E/F}| with |W_{E/F}|>0°. The angle range from 180° to 180°−|W_{E/F}| corresponds to a dorsal extension, the angle range from 180° to 180°+|W_{E/F}| corresponds to a palmar flexion. The angles of the ulnar and radial deviation, also referred to as “ulnar abduction” and “radial abduction”, can accordingly be advantageously either fixed at |W_{D1}|=|W_{D2}|=0° (hand pointing straight forward) or released in a controlled manner in a range of movement of W_{D1|}, |W_{D2}|>0°. The values of the angles |W_{D1}| and |W_{D2}| can be the same size but also different sizes, with the result that an asymmetrical movement framework can be specified. The axes of movement for the extension and flexion movement or for the ulnar and radial deviation, which are technically realized by the movement module of the orthosis joint according to the invention, are advantageously located in the region of the physiological joint axes and in this way represent the “natural” range of movement of the wrist or hand particularly satisfactorily.

An orthosis according to the invention with such an adjustable orthosis joint can advantageously accompany the healing process in such a way that the freedom of movement of the patient can be increased in a controlled manner, wherein movements which are permitted from a therapeutic point of view can be guided in a controlled manner and at the same time movements which are critical from a therapeutic point of view can be completely or partially restricted. In this way, unnecessary stiffening of healthy joints of the hand can be advantageously counteracted. The orthosis joint according to the invention has a particularly compact construction, with the result that an orthosis according to the invention equipped therewith can advantageously likewise be of compact configuration and provides the least hindrance to the patient in their everyday life.

Further advantageous refinements and developments which can be used individually or in combination with each other are the subject matter of the dependent claims.

In one refinement of the invention, it has proven to be useful if the control element comprises at least one first control slide, which can be moved by means of at least one first actuating means with respect to the pivot lever which is operatively connected to the movement module. With the aid of such a first control slide, the operative connection between the movement module and the pivot lever can advantageously be changed safely and easily for controlling the extension and flexion movement of the hand, and can thus be adjusted to the individual needs of the patient.

It is advantageous if the first control slide is of substantially cuboid-shaped configuration and, at its end facing the pivot lever, is of fork-shaped configuration. A displaceable, cuboid-shaped first control slide with a fork-shaped end can be advantageously pushed up to the pivot lever in such a way that in particular the end of the pivot lever, which can be pivotally mounted by means of a pivot axis, is surrounded by the “fork arms”, and the end of the pivot lever is thus located in the interior of the fork-shaped end of the first control slide. The “fork arms”, i.e. the top and bottom parts of the fork-shaped end, can advantageously each act as a stop and limit the extent of the pivot movement of the pivot lever.

In a further preferred refinement of the invention, it has proven to be useful if the first actuating means comprises at least one head and one actuating cylinder which are connected to each other, preferably via a collar, with the result that a component with a longitudinal axis is formed, wherein the actuating cylinder has a circular cross section and is connected to the head of the first actuating means eccentrically with respect to the longitudinal axis. The first control slide can then advantageously comprise at least one cutout which is configured to receive the actuating cylinder and to interact with it. A first control means with an actuating cylinder, which has a circular cross section and is connected eccentrically to the head, can, when the first control means is received by the cutout of the first control slide, move it by virtue of the fact that the actuating cylinder passes into an operative connection with a side boundary of the cutout during rotation of the first actuating means, exerts force on said side boundary, and thus pushes the first control slide away from the rotational axis of the first actuating means. Due to the eccentric arrangement of the actuating cylinder in the sense of an eccentric screw, the operative connection to said side boundary decreases when the first actuating means is rotated in the opposite direction, and increases to an opposite side boundary of the cutout, with the result that the first control slide is pushed accordingly in the other direction.

As an alternative to this, one refinement of an orthosis joint has proven to be useful, in which the first actuating means comprises at least one head and one actuating cylinder which are connected to each other, preferably via a collar, with the result that a component with a longitudinal axis is formed, wherein, on its side which faces away from the head, the actuating cylinder has a channel which is preferably substantially C-shaped. The first control slide can then accordingly comprise a connecting pin which engages into the channel of the actuating cylinder during use of the orthosis joint, wherein the connecting pin is preferably arranged within a cutout of the first control slide, which is configured to receive the actuating cylinder. In the case of a rotation of such a designed first actuating means, force can be advantageously exerted on the first control slide via the connecting pin, which engages in the substantially C-shaped channel of the actuating cylinder, by interaction with the walls of the channel. Depending on the direction of rotation, the first control slide can thus be advantageously moved in the direction of the pivot lever or in the opposite direction.

In addition, one refinement has proven itself, in which the first actuating means and/or the first control slide comprises at least one snap-in mechanism which is configured to reversibly fix the first control slide in its displacement movement with respect to the pivot lever which is operatively connected to the movement module at at least one predetermined position. A snap-in mechanism advantageously enables predetermined maximum possible extension/flexion angles to be set safely and quickly.

In a further preferred refinement of the invention, the control element can comprise a first cutout for deviation control, which is arranged on the bottom side of the control element; and which extends from an edge of the control element in the direction of the rotary cylinder, with the result that a height of the control element in the region of the first cutout is smaller than a height of the control element in the region outside the first cutout. As a result, at least four side edges are formed which delimit the region within the first cutout from the area outside the first cutout, of which two lie substantially opposite each other at a spacing dₙ where n=1, 2, . . . and form a side edge pair SPₙ where n=1, 2, . . . ; wherein a spacing dₙ between two side edges, which are arranged closer to the rotary cylinder and lie opposite one another, of a side edge pair SPₙ is always smaller than a spacing dₙ₊₁ between two side edges, which are arranged closer to the edge of the control element and lie opposite one another, of a side edge pair SPₙ₊₁. A control element which is configured in this way advantageously allows the adjustment and control of at least two different maximum rotation angles around the deviation axis, thus a control of the angles of the ulnar and radial deviation. One refinement of the invention is particularly preferred here, in which a multiplicity of side edges, preferably eight side edges, are formed, of which two each lie substantially opposite each other and form a side edge pair SPₙ where n=1, 2, . . . . The more side edge pairs SPₙ are provided, the smaller the steps into which the angular ranges of ulnar and radial deviation can be divided by the patient and/or therapist. The provision of four side edge pairs SPₙ (corresponding to eight side edges) for setting maximum ulnar/radial deviation movements of |W_{D1}| and |W_{D2}|=0°, 10°, 20°, 30°has proved to be particularly suitable for generating as fast a rehabilitation progress as possible, but any other desired angles can also be provided.

In this regard, one refinement having a second control slide, which interacts with the control element, for the control of the ulnar/radial deviation movement of the hand has proven itself, wherein the second control slide is of substantially cuboid-shaped configuration and has a length, a width and a height. Here, the width can preferably correspond to the spacing d₁ of the opposite side edges of the first side edge pair SP₁ arranged closest to the rotary cylinder; and the second control slide can be configured such that it can be inserted into the first cutout of the control element and can be removed from it and can interact with the side edges. A second control slide having a width which corresponds to the spacing d₁ of the opposite side edges of the first side edge pair SP₁ arranged closest to the rotary cylinder, advantageously allows a complete fixation of the hand in a straight position with respect to a deviation movement, so in a position with W_{D1}| and W_{D2}|=0°, especially when the second control slide has been pushed completely onto the rotary cylinder.

As an alternative to this, a further refinement of the orthosis joint according to the invention has proven to be useful, in which two second control slides which interact with the control element for the control of the ulnar/radial deviation movement of the hand are provided, wherein the second control slides are each of substantially cuboid-shaped configuration and have a length, a width and a height; wherein the sum of the widths corresponds to the spacing d₁ of the opposite side edges of the first pair of side edges SP1 arranged closest to the rotary cylinder. Here, each second control slide can be configured such that it can be inserted into the first cutout of the control element and can be removed from it and can interact with the side edges Two second control slides, the widths of which add up to the spacing d₁ of the opposite side edges of the first side edge pair SP₁, arranged closest to the rotary cylinder, advantageously allow the total range of movement of the deviation movement of the hand to be divided into an angular range of the ulnar abduction (corresponding to |W_{D2}|>0°) and into an angular range of radial abduction (corresponding to |W_{D1}|>0°) and to be controlled independently of each other. Both movements in an angular range of 0°≤|W_{D2}|≤30° for fixation or controlled ulnar abduction, movements in an angular range of 0°≤|W_{D1}|≤30° for fixation or controlled radial abduction, and a combination of different angles |W_{D1}|, |W_{D2}| can advantageously be set by the patient or therapist.

In addition, it has proven itself in a further preferred refinement if each second control slide is configured to be reversibly fixed at predetermined positions during insertion into and removal from the first cutout, preferably by means of a snap-in mechanism. To this end, each second control slide can have a sawtooth profile, in particular on its side directed toward the orthosis, which can pass reversibly into operative connection with a correspondingly configured side, directed toward the second control slide, of the orthosis and/or a housing for the second control slide or slides and can thus form a snap-in mechanism. Alternatively or cumulatively, the snap-in mechanism can also be formed by one or more, preferably spring-loaded, pins or studs, which can be arranged on the side, directed toward the second control slide, of the orthosis and/or the housing for the second control slide and interact with correspondingly formed depressions, in particular holes, in that side of the second control slide or slides which is directed toward the orthosis. One or two control slides configured in this way advantageously enable predetermined deviation angles for a maximum possible ulnar and/or radial abduction to be set safely, quickly and without the use of tools.

In a further alternative refinement of the orthosis joint according to the invention, two second control slides which interact with the control element for the control of the ulnar/radial deviation movement of the hand and at least one fourth cutout which is arranged on the control element for the deviation fixation can be provided.

In this particularly preferred embodiment, one of the second control slides may be formed as a deviation control slide and at least one head region which is arranged in the first cutout of the control element to be inserted and led out and to interact with the side edges of the first cutout of the control element, and a body region, which comprises a guide track for a control pin of a second actuating means comprising the orthosis joint. The other of the two second control slides can, in contrast, be designed as a fixing slide, and can comprise at least a head region which is configured to interact with the fourth cutout of the control element, and a body region which has a guide track for the control pin of the second actuating means. In this refinement, the deviation control slide and the fixing slide are additionally preferably displaceably mounted and configured in such a way that they can be displaced by means of the control pin of the second actuating means with respect to each other.

Two second control slides of such configuration, that is to say one second control slide configured as a deviation slide and one second control slide configured as a fixing slide, which can be moved with respect to each other by means of a second control means, advantageously allow both the control of the ulnar and radial deviation of the hand and a fixation of the deviation movement at a certain angle by means of a single further component, namely by means of the second actuating means. The adjustment and fixation of the ulnar and radial deviation can be advantageously achieved in this refinement via the rotation of the second actuating means, and thus, in a similarly simple manner to the adjustment and fixation of the extension and flexion movement of the hand, via rotation of the first actuating means, without the use of further, special tools.

In addition, it has proven to be useful if the head region of the deviation control slide is formed by two opposite lever elements, which are resiliently movable with respect to each other and the outer edges of which are at a spacing from each other in a relaxed state, which spacing corresponds to the spacing d₁ of the opposite side edges of the first side edge pair SP₁, arranged closest to the rotary cylinder, of the first cutout. The head region of the fixing slide can additionally preferably comprise a head element for interaction with the fourth cutout, which is firmly connected to the body region of the fixing slide. The head region of a deviation control slide of such configuration can advantageously dampen the interaction of the outer edges of the deviation control slide with the side edges of the first cutout and can thus impart the user the feeling of a softer stop in the respective end position of the deviation movement. The head region of a fixing slide of such configuration can advantageously interact with the control element, in particular with one or more fourth cutouts, which are arranged on the control element, preferably within the first cutout of the control element, and can thus allow a fixation of the deviation movement of the hand in a desired angular position.

The invention also relates to an orthosis for the controlled movement and/or fixation of a hand or a hand and a forearm of a patient, with an adjustable orthosis joint as described above.

These and additional details and further advantages of the invention are described below on the basis of preferred exemplary embodiments, to which the present invention is not limited, however, and in conjunction with the accompanying drawing,

in which, diagrammatically:

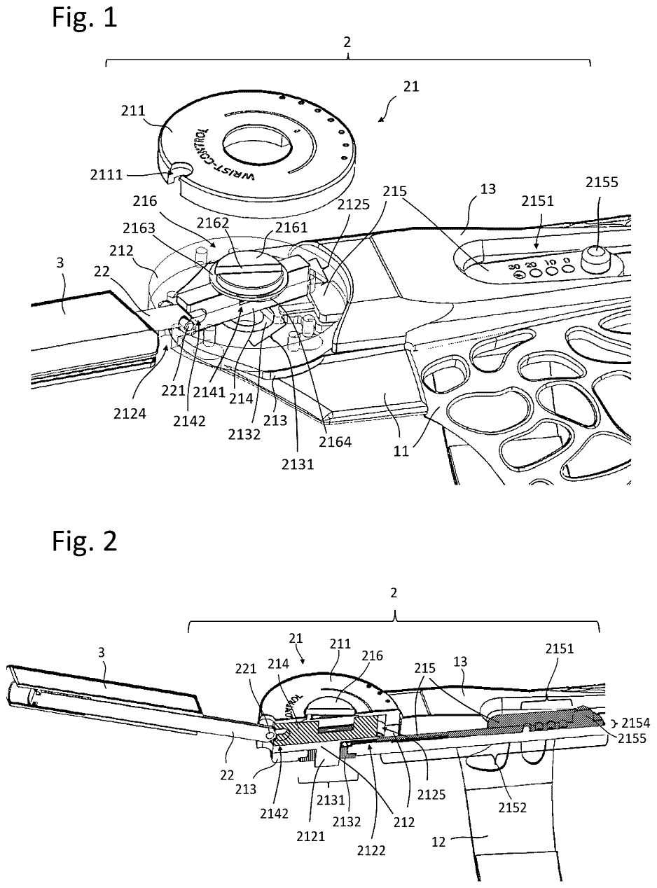

FIG. 1 shows one embodiment of an orthosis joint according to the invention in a perspective view (control element shown as an outline drawing),

FIG. 2 shows a section along a longitudinal axis of an orthosis comprising an embodiment of an orthosis joint according to the invention,

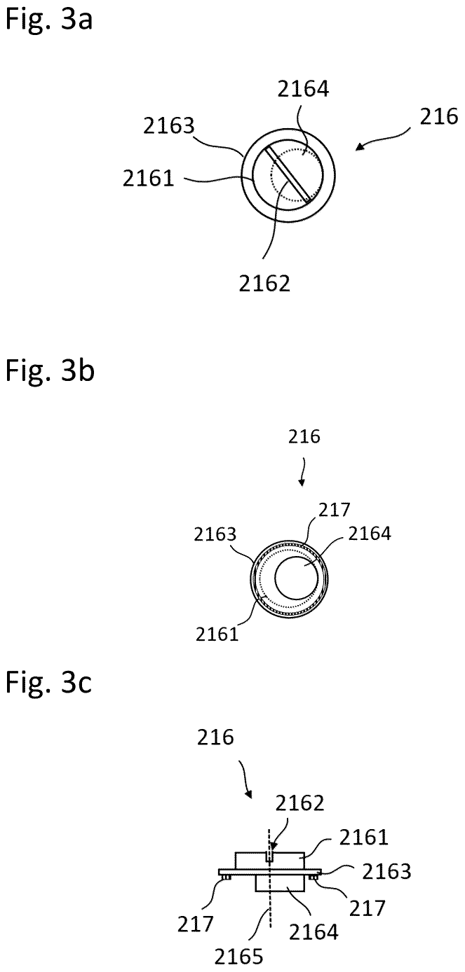

FIG. 3 shows, in subfigures a to c, one embodiment of a first actuating means according to the invention in a plan view (FIG. 3a), with a view of a bottom side of the first control means (FIG. 3b) and in a side view (FIG. 3c),

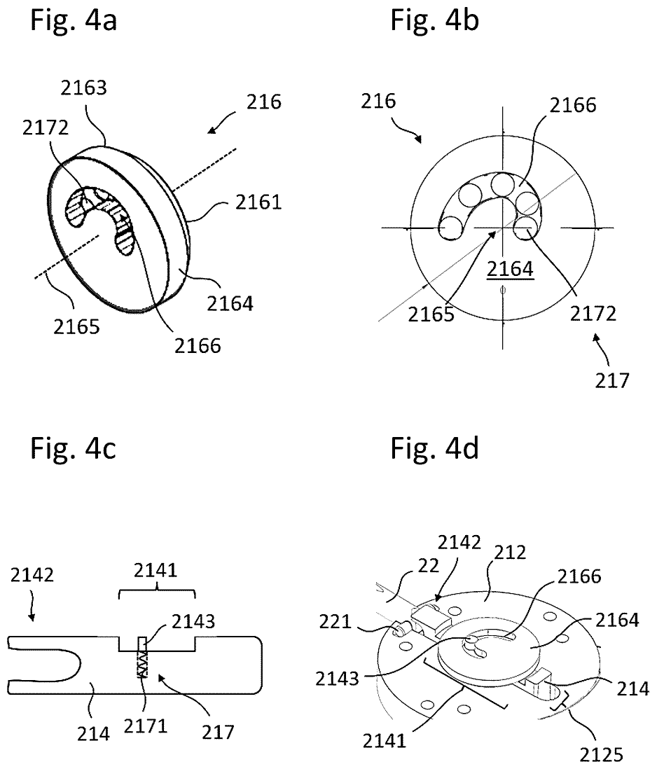

FIG. 4 shows a part drawing of a further embodiment of a first actuating means according to the invention in a perspective view of a bottom side of the first actuating means (FIG. 4a) and in a view with a view of a bottom side of the first actuating means (FIG. 4b), a first control slide of corresponding configuration with respect thereto in a side view (FIG. 4c) as well as a perspective view of a control element with such a first actuating means of such configuration (FIG. 4d),

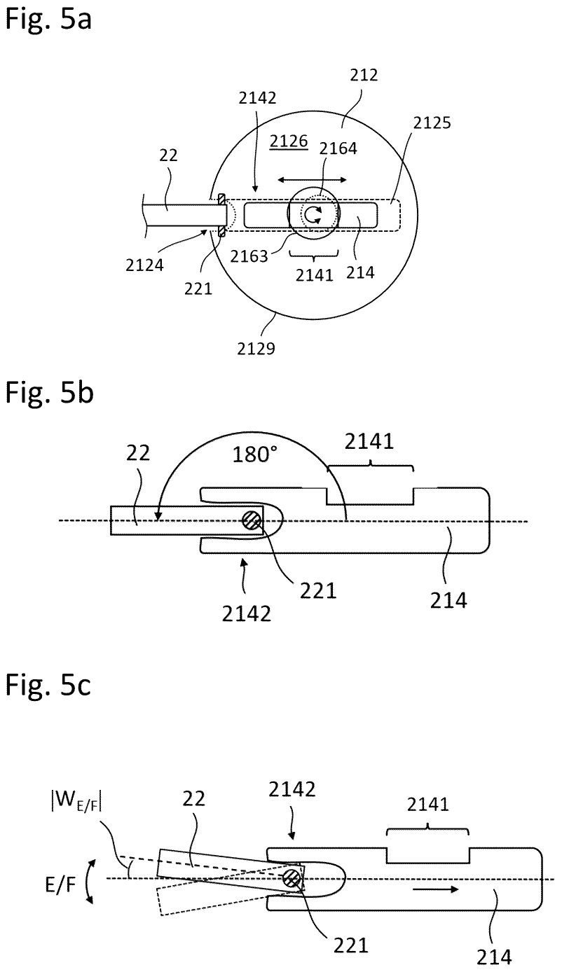

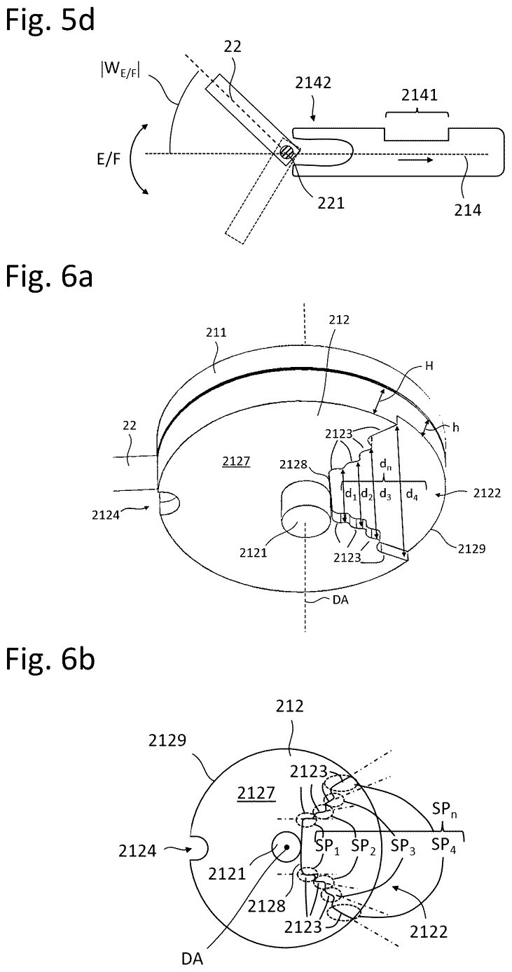

FIG. 5a shows a plan view of one embodiment of a control element with one embodiment of a first control slide and a pivot lever which is operatively connected to it,

FIGS. 5b-d show, in subfigures 5b to 5d, in each case a side view of three possible positions of the first control slide in relation to the pivot lever for illustration of the mode of action of the first control slide in the control of the extension and flexion of the hand,

FIG. 6a shows one embodiment of a control element and a cover element in a perspective view of the bottom side of the control element,

FIG. 6b shows the control element from FIG. 6a with a view of the bottom side,

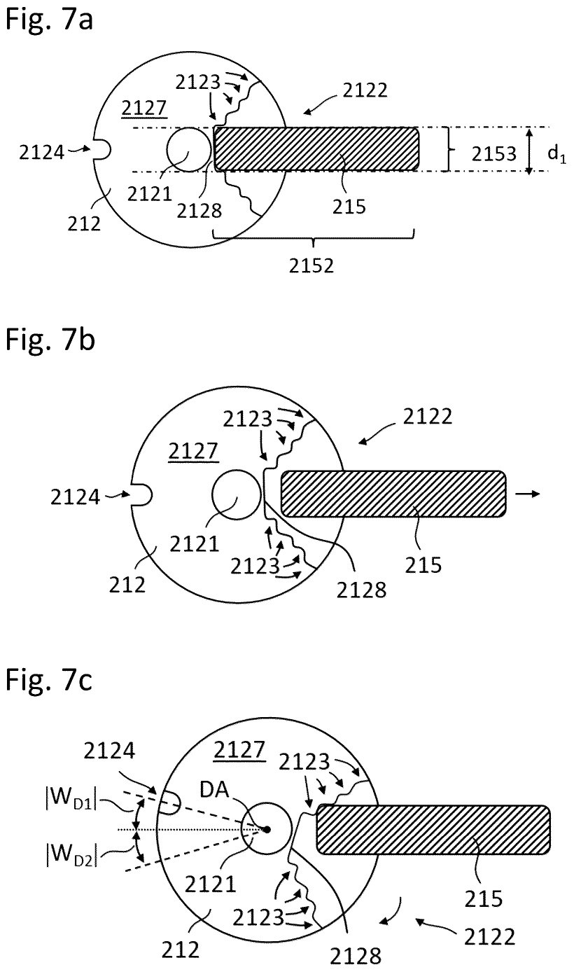

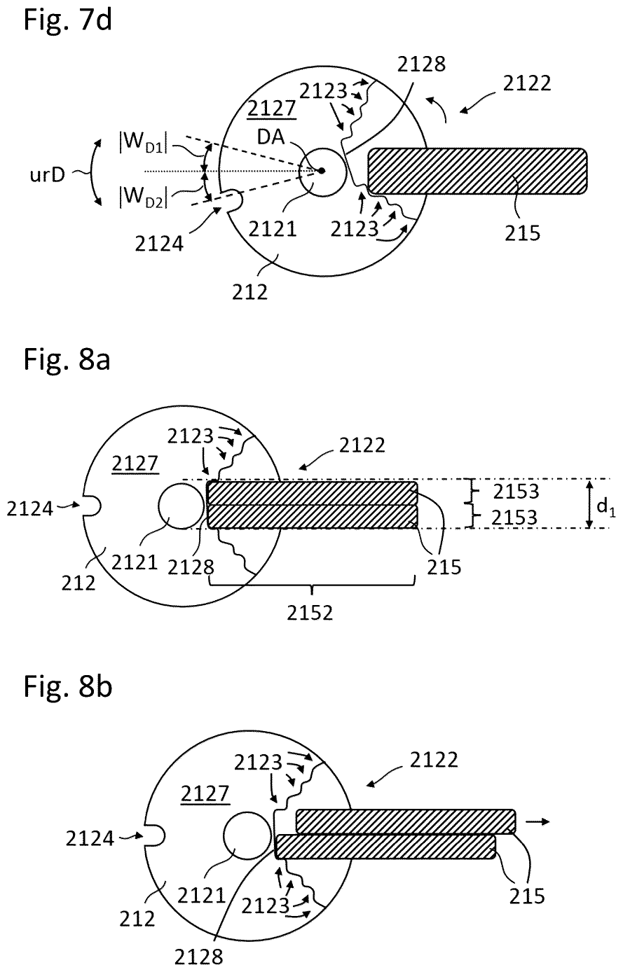

FIG. 7 shows, in subfigures a to d, the interaction of one embodiment of a second control slide with the control element from FIGS. 6 a and b for illustration of the control of the deviation movement of the hand,

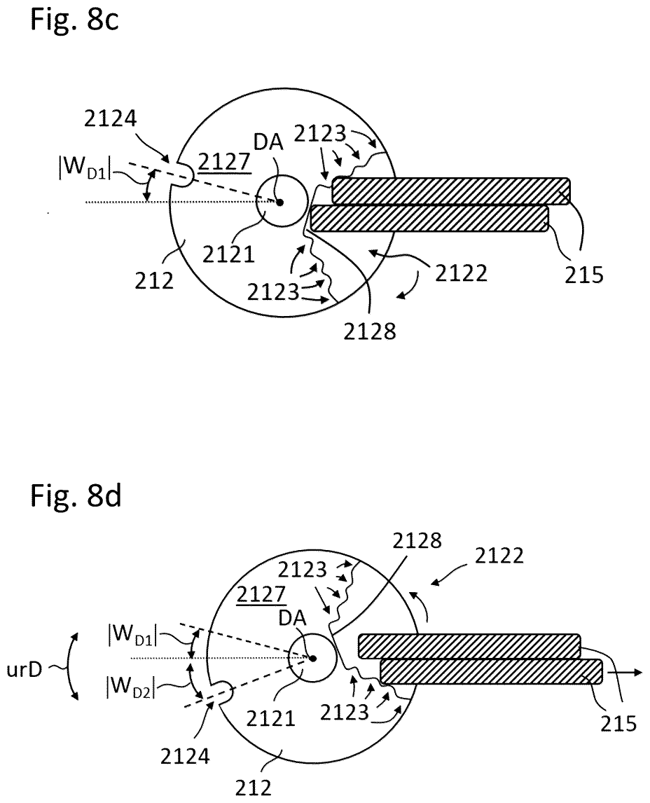

FIG. 8 shows, in subfigures a to d, the interaction of two second control slides with the control element from FIG. 6 a and b for illustration of the control of the deviation movement of the hand,

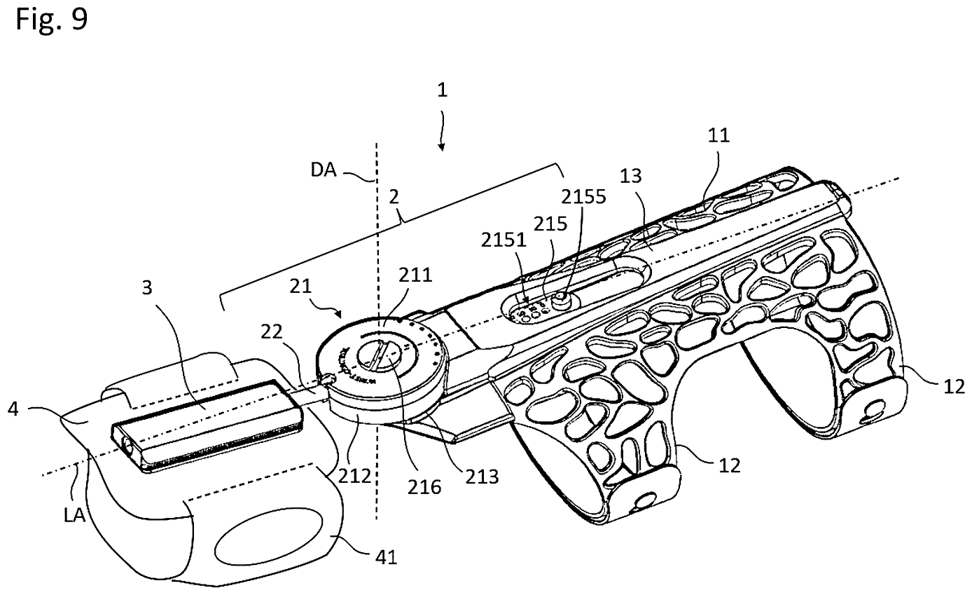

FIG. 9 shows one embodiment of an orthosis with one embodiment of an orthosis joint according to the invention in a perspective view,

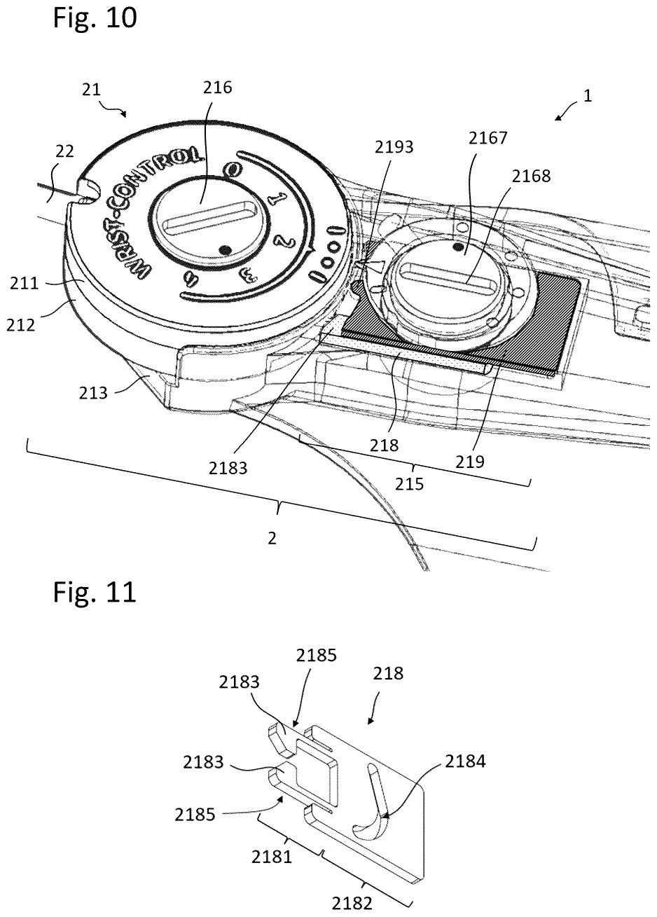

FIG. 10 shows a further embodiment of an orthosis joint according to the invention with two second control slides, wherein one of the control slides is configured as a deviation control slide and the other as a fixing slide,

FIG. 11 shows one embodiment of a deviation control slide as shown individually in FIG. 10,

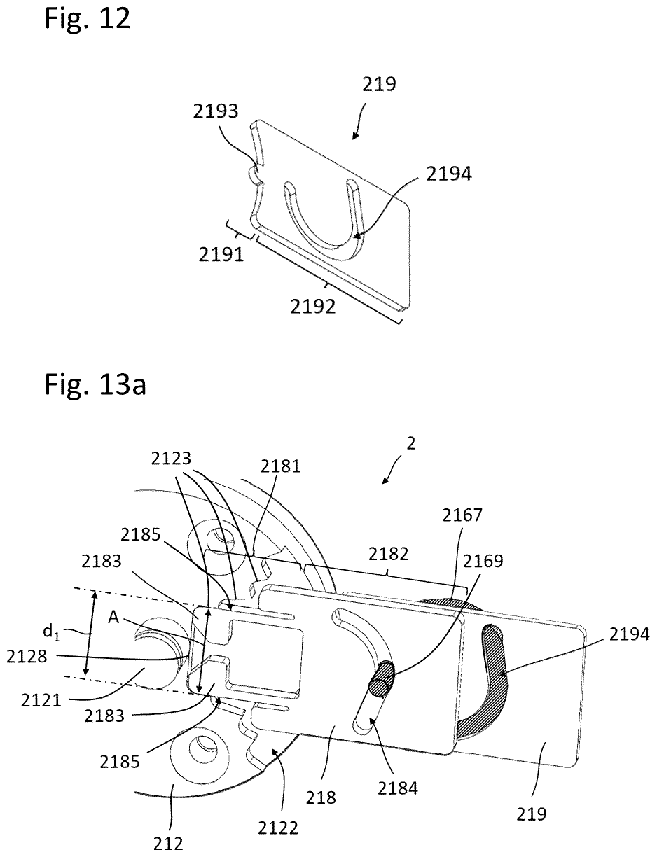

FIG. 12 likewise individually shows one embodiment of a fixing slide as shown in FIG. 10,

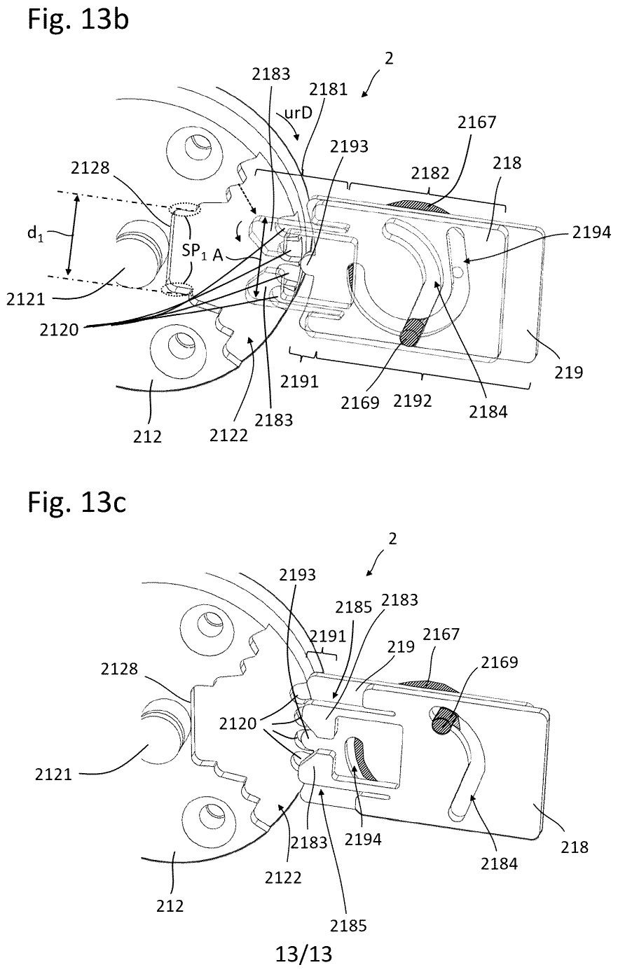

FIG. 13 shows, in subfigures a to c, the interaction of the embodiment of an orthosis joint shown in FIG. 10 with a deviation control slide and a fixing slide for illustrating the control and fixation of the deviation movement of the hand, and

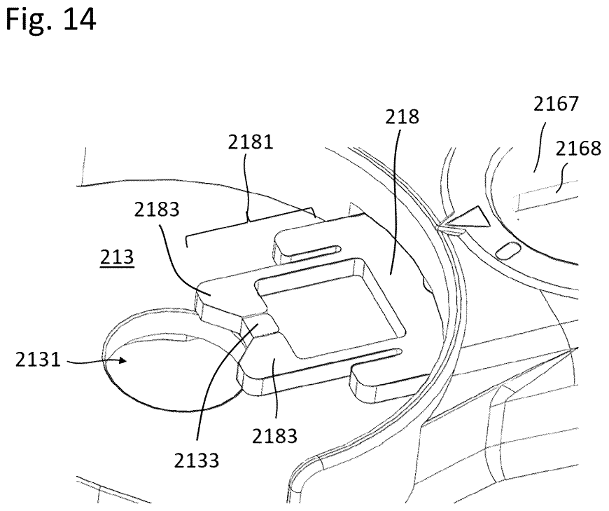

FIG. 14 shows a view of one embodiment of a base element of an orthosis joint as shown in FIG. 10 and a deviation control slide inserted therein.

In the following description of preferred embodiments of the present invention, the same designations denote identical or comparable components.

In FIG. 1, one embodiment of an orthosis joint 2 according to the invention is shown in a perspective view. A control element 212 is shown as an outline drawing in order to make components underneath visible.

An adjustable orthosis joint 2 according to the invention for controlled movement and/or fixation of a patient's hand comprises at least one movement module 21 for controlling an extension and/or flexion movement E/F and for controlling and executing an ulnar and/or radial deviation urD of the hand. To carry out the extension and/or flexion movement E/F of the hand, a pivot lever 22 is operatively connected to the movement module 21. To this end, the pivot lever 22 can preferably be operatively connected to the control element 212 via a pivot axis 221 or can be pivotably mounted thereon. The movement module 21 further comprises at least one base element 213 and at least one control element 212 having a top side 2126 and a bottom side 2127 facing the base element 213 and lying opposite the top side 2126. On the bottom side 2127, a rotary cylinder 2121 is arranged, by means of which the control element 212 is rotatably mounted relative to the base element 213 with respect to a deviation axis DA. The base element 213 may preferably have a mount 2131 for the rotary cylinder 2121 to this end. The control element 212 together with the rotary cylinder 2121 may be manufactured in particular from a metallic material, preferably from aluminum. For the rotatable mounting of the control element 212 relative to the base element 213, a guide means 2132 for the rotary cylinder 2121 can be provided, moreover, between the base element 213 and the rotary cylinder 2121. Such a guide means 2132, which is preferably manufactured from steel in the form of a sleeve, can advantageously reduce friction of the metallic rotary cylinder 2121 relative to the base element 213, in particular with respect to a base element 213 manufactured from a plastic (see in particular FIG. 2 in this regard).

According to the invention, the control element 212 is designed to be limited in its rotational movement around the deviation axis DA in a controlled manner, and the control element 212 is also designed to limit the movement of the pivot lever 22 in its range of movement in a controlled manner. The pivot axis 221, via which the pivot lever 22 is preferably operatively connected to the control element 212, can be arranged for this purpose within a cutout 2124, which in turn can be arranged at the edge 2129 of the control element 212. This second cutout 2124 may extend in particular over an entire height H of the control element 212, and thus allow the pivot lever 22 freedom of movement for its pivot movement perpendicularly with respect to the plane of the control element 212, and is preferably arranged on the opposite edge 2122 to a first cutout 2129, arranged on the bottom side 2127 of the control element 212, for deviation control. Accordingly, a cutout 2111 for the pivot lever 22 can also be provided in a cover element 211 for covering the control element 212. As can be seen in FIG. 1, the control element 212 can also comprise at least one first control slide 214, which is movable by means of at least one first actuating means 216 with respect to the pivot lever 22 which is operatively connected to the movement module 21. To this end, the control element 212 may have a third cutout 2125 for the first control slide 214, which is preferably arranged on the top side 2126 of the control element 212 and in particular extends along a connecting line from the center of the first cutout 2122 for the deviation control to the center of the second cutout 2124 for the pivot lever 22.

FIG. 2 shows a section along a longitudinal axis LA of an orthosis 1 comprising one embodiment of an orthosis joint 2 according to the invention. In FIG. 2, the first control slide 214 is highlighted in a diagonally hatched manner and it can be seen how it is guided within the third cutout 2125.

The first control slide 214 can, as shown in FIGS. 1 and 2, be of substantially cuboid-shaped configuration, and can be of fork-shaped configuration at its end facing the pivot lever 22. The fork-shaped end 2142 of the first control slide 214 may preferably be configured such that that end of the pivot lever 22 which is connected to the pivot axis 221 can be pushed between the “fork arms”, wherein the scope of the pivot movement of the pivot lever 22 is all the more restricted, the further that end of the pivot lever 22 which is connected to the pivot axis 221 is received into the space between the arms of the fork-shaped end 2142. FIGS. 5b to 5d illustrate this mechanism by way of example. The first actuating means 216 provided for the movement of the first control slide 214 within the third cutout 2125 preferably comprises at least one head 2161 and one actuating cylinder 2164, which are connected to each other, preferably via a collar 2163, with the result that a component with a longitudinal axis 2165 is formed. The collar 2163 can be used in particular to fix the first actuating means 216 within the movement module 21 rotatably, to which end the movement module 21 can comprise a cover element 211, which at least partially covers the control element 212 together with all the components arranged thereon toward the top, that is to say on that side of the movement module 21 which lies opposite the base element 213. In FIG. 2 it can be seen, for example, that the cover element 211 covers the control element 212 and all components arranged thereon apart from the head 2161 of the first actuating means 216 toward the top. The collar 2163 is located here below the cover element 211 and prevents the first actuating means 216 from falling out of the opening in the middle of the cover element 211, which is easily recognizable in FIG. 1.

FIG. 3 shows, in subfigures a to c, one embodiment of a first actuating means 216 according to the invention in a plan view (FIG. 3a), with a view of a bottom side of the first actuating means 216 (FIG. 3b) and in a side view (FIG. 3c).

The actuating cylinder 2164 of the first actuating means 216 can—as shown here—have a circular cross section and can be connected eccentrically with respect to the longitudinal axis 2165 to the head 2161 of the first actuating means 216. The radius of such an actuating cylinder 2164 may in particular be smaller than the radius of the head 2161. The actuating cylinder 2164 can also interact with at least one cutout 2141 of the first control slide 214, which is configured for receiving the actuating cylinder 2164. To this end, the first actuating means 216 can be rotated by the patient themselves via a groove 2162, which is preferably arranged on the top side of the head 2161, whereby the actuating cylinder 2164 passes into an operative connection to a side boundary of the cutout 2141, exerts force on said side boundary and thereby pushes the first control slide 214 away from the rotational axis of the first actuating means 216. Due to the eccentric arrangement of the actuating cylinder 2164 in the sense of an eccentric screw, the operative connection to said side boundary decreases when the first actuating means 216 is rotated in the opposite direction, and increases to an opposite side boundary of the cutout 2141, with the result that the first control slide 214 is pushed correspondingly in the other direction.

In FIG. 4, a further embodiment of a first actuating means 216 according to the invention is shown in a part drawing in a perspective view of a bottom side of the first actuating means 216 (FIG. 4a) or in a view looking at a bottom side of the first actuating means 216 (FIG. 4b), as well as a first control slide 214 correspondingly configured with respect thereto in a side view (FIG. 4c) and a perspective view of a control element 212 with a first actuating means 216 of such configuration (FIG. 4d). In FIG. 4d, the first actuating means 216 is not shown completely, but rather merely a section through the actuating cylinder 2164 of a first actuating means 216 in FIGS. 4a and 4b is shown, in order to illustrate the engagement and the guidance of the connecting pin 2143 within the channel 2166.

As an alternative to the above-described embodiment, the first actuating means 216 may also comprise at least one head 2161 and one actuating cylinder 2164, which, preferably via a collar 2163, are connected to each other, with the result that a component with a longitudinal axis 2165 is formed, wherein the actuating cylinder 216 has, on its side facing away from the head 2161 a preferably substantially C-shaped channel 2166. In this embodiment, the first control slide 214 may in particular comprise a connecting pin 2143, which engages into the channel 2166 of the actuating cylinder 2164 during use of the orthosis joint 2. The connecting pin 2143 can preferably be arranged within a cutout 2141 of the first control slide 214, which is configured to receive the actuating cylinder 2164 (see FIG. 4c). The head 2161 of the first actuating means 216 can be provided as before with a groove 2162, via which the first actuating means 216 can be rotated with respect to its longitudinal axis 2165. In a rotation of a first actuating means 216 of such configuration, force can be advantageously exerted on the first control slide 214 via the connecting pin 2143, which engages into the substantially C-shaped channel 2166 of the actuating cylinder 216, by interaction with the walls of the channel 2166 (FIG. 4d). Depending on the direction of rotation, the first control slide 214 can thus be moved in the direction of the pivot lever 22 or in the opposite direction within the third cutout 2125 of the control element 212.

To illustrate the mode of action of the first control slide 214 in the control of the extension and flexion of the hand, FIG. 5a now shows a plan view of one embodiment of a control element 212 with one embodiment of a first control slide 214 and a pivot lever 22 which is operatively connected thereto, and partial FIGS. 5b to 5d in each case show a side view of three possible positions of the first control slide 214 in relation to the pivot lever 22 during use.

As already described, the first control slide 214, guided by the third cutout 2125 provided on the control element 212, can be moved with respect to the pivot lever 22 or its pivot axis 221 with the aid of the first actuating means 216. By way of example, a circular, eccentrically positioned actuating cylinder 2164 is indicated using dashed lines in FIG. 5a; an actuating cylinder 2164 with channel 2166, as previously described (see FIGS. 4a and 4b), however, acts accordingly. FIG. 5b now shows a position of the first control slide 214, in which it is pushed in completely to the pivot axis 221 of the pivot lever 22, and the fork-shaped end 2142 of the first control slide 214 completely surrounds said pivot axis 221 and the adjacent part of the pivot lever 22. The fork arms of the fork-shaped end 2142 act as a stop and prevent a pivoting movement of the pivot lever 22 around its pivot axis 221. The angle between the pivot lever 22 and the first control slide 214 is 180° in this position (indicated in FIG. 5b by an angle arc and a dashed line), which corresponds to a hand of a patient extended with respect to the forearm and represents, so to speak, a zero position. In this position of the first control slide 214, the hand is thus fixed with respect to an extension and flexion movement E/F. FIG. 5c shows a position of the first control slide 214, in which it has been pushed away by means of the first actuating means 216 from the pivot axis 221 of the pivot lever 22. This movement is indicated by an arrow pointing to the right in FIG. 5c. In this position, the fork-shaped end 2142 of the first control slide 214 still grips that part of the pivot lever 22 located near the pivot axis 221 to a certain degree, but no longer prevents the pivot movement of the pivot lever 22 completely. The fork arms in turn serve as a stop and limit the extension and flexion movement E/F of the hand guided by the pivot lever 22 at a predetermined angle ±W_{E/F}. The double amount |W_{E/F}| of this angle then determines the maximum range of movement possible for the extension and flexion movement E/F of the patient's hand. FIG. 5d shows the first control slide 214 finally in a position moved completely to that end of the third cutout 2125 which lies opposite the pivot axis 221. The fork-shaped end 2142 no longer grips the pivot lever 22 and its pivot axis 221. The pivot movement of the pivot lever 22 and thus also the extension and flexion movement E/F of the hand takes place in a completely unhindered manner in the anatomically possible range of movement. By the operative connection of the pivot lever 22 to the control element 212 via the pivot axis 221, however, guidance of the extension and flexion movement E/F of the hand can still advantageously take place and thus the wrist of the patient can be stabilized.

The first actuating means 216 and/or the first control slide 214 can, in particular to ensure an adjustment, which is safe and can be carried out by the patient themselves, of the respective possible range of movement of the extension and flexion movement E/F of the hand, preferably comprise at least one snap-in mechanism 217, which is configured to reversibly fix the first control slide 214 in its displacement movement with respect to the pivot lever 22, operatively connected to the movement module 21, at at least one predetermined position. FIG. 4a to d show one embodiment of such a snap-in mechanism 217 using the example of a first actuating means 216 with channel 2166. To this end, the channel 2166 arranged on that side of the actuating cylinder 2164 which faces away from the head 2161 can include depressions 2172, which are arranged at predetermined positions within the channel 2166. The connecting pin 2143 arranged on the first control slide 214 can then engage into these depressions 2172 when the first actuating means 216 is rotated about its longitudinal axis 2165, but can also be moved out of the respective depression 2172 again when applying a small additional force, and can be guided as far as the next depression 2172. The patient can feel the “snap” into the individual depressions 2172 when rotating the first actuating means 216. It thus advantageously signals them a correct adjustment of the orthosis joint 2. In one preferred embodiment of such a snap-in mechanism 217, the connecting pin 2143 can also be mounted in a spring-loaded manner on the first control slide 214. In FIG. 4c, a spring 2171 is shown as an example in this respect. Said spring 2171 can advantageously facilitate both the engagement and the release of the connecting pin 2143 into/from a depression 2172 within the channel 2166. In one embodiment of the first actuating means 216 with an eccentrically arranged actuating cylinder 2164 as shown in FIG. 3, the snap-in mechanism 217 may be designed in particular as a structured, preferably corrugated ring, which is arranged on that side of the collar 2163 which faces the actuating cylinder 2164, and interacts with a correspondingly structured region of the control means 212 and/or the first control slide 214.

FIGS. 6a and 6b show one embodiment of a control element 212 and a cover element 211 in a perspective view of the bottom side 2127 of the control element 212 (FIG. 6a); and a view of said bottom side 2127 (FIG. 6b).

The control element 212, as previously mentioned, can preferably comprise a first cutout 2122 for the deviation control, which is arranged on the bottom side 2127 of the control element 212 and extends from an edge 2129 of the control element 212 in the direction of the rotary cylinder 2121. A height h of the control element 212 can thus be smaller in the region of the first cutout 2122 than a height H of the control element 212 in the region outside the first cutout 2122, whereby at least four side edges 2123 are formed which delimit the region within the first cutout 2122 from the region outside the first cutout 2122. Two of the side edges 2123 can each lie substantially opposite each other at a spacing dₙ where n=1, 2, . . . and form a side edge pair SPₙ where n=1, 2, . . . . A spacing dₙ between two opposite side edges 2123, arranged closer to the rotary cylinder 2121, of a side edge pair SPₙ is preferably always smaller than a spacing dn₊₁ between two opposite side edges 2123, arranged closer to the edge 2129 of the control element 212, of a side edge pair SPₙ₊₁. As can be seen in FIG. 6a and b, the first cutout 2122 can also be formed by a multiplicity of side edges 2123, preferably eight side edges 2123, of which two each lie substantially opposite each other and form a side edge pair SPₙ where n=1, 2, . . . . Toward the rotary cylinder 2121, the first cutout 2122 may be closed in particular by an end edge 2128. The opposite side edges 2123 of the first side edge pair SP₁, arranged adjacently with respect to the rotary cylinder 2121, can, as shown in FIG. 6b, run approximately parallel to a connecting line, which connects the center of the second cutout 2124 for the pivot lever 22 and the intersection point of the deviation axis DA extending through the rotary cylinder 2121 with the plane of the control element 212 to each other. The side edges 2123 of the side edge pairs SP₂, SP₃, SP₄, . . . preferably form an angle with this connecting line. In FIG. 6b, the individual side edges 2123 are extended by dash-dotted lines to illustrate the respective resulting angle.

To control the ulnar and radial deviation movement urD of the hand, a second control slide 215 can now interact with the control element 212. The second control slide 215 can be of substantially cuboid-shaped configuration to this end and can have a length 2152, a width 2153 and a height 2154. The width 2153 of the second control slide 215 preferably corresponds to the spacing d₁ of the opposite side edges 2123 of the first side edge pair SP₁, arranged closest to the rotary cylinder 2121. The second control slide 215 can also be configured such that it can be inserted into the first cutout 2122 of the control element 212 and such that it can be removed from it, and can preferably interact with the side edges 2123.

FIG. 7 shows, in subfigures a to d, the interaction of one embodiment of a second control slide 215 with the control element 212 from FIGS. 6a and b for illustration of the control of the deviation movement of the hand.

FIG. 7a diagrammatically shows the bottom side 2127 of the control element 212 with rotary cylinder 2121, first cutout 2122 and second cutout 2124 in a plan view. The second control slide 215 is inserted into the first cutout 2122 as far as the end edge 2128 of the first cutout 2122. The width 2153 of the control slide 215 corresponds in this embodiment to the spacing d₁ between the opposite side edges 2123 of the first side edge pair SP₁. In this position, the edges of the second control slide 215 thus interact with the side edges 2123 of the first side edge pair SP₁, with the result that a rotation of the control element 212 about the deviation axis DA extending through the rotary cylinder 2121 is prevented. A pivot lever 22 (not shown here, but can be seen e.g. in FIGS. 1, 2, 5 and 9) which is operatively connected to the control element 212 can thus likewise not move within the framework of a rotational movement with respect to the deviation axis DA, with the result that no deviation movement of the hand is permitted-the hand is fixed with respect to an ulnar and radial deviation urD.

In FIG. 7b, the second control slide 215 is shown in a position in which it is guided a little out of the first cutout 2122 and is guided away from the end edge 2128 of the first cutout 2122. In this position, there is no longer contact with the side edges 2123 of the first side edge pair SP₁, and a rotational movement of the control element 212 by means of the rotary cylinder 2121 about the deviation axis DA is possible (curved arrows in FIGS. 7c and 7d). In FIGS. 7c and 7d, the end points of said rotational movements are shown, wherein FIG. 7c shows the end point of a radial deviation movement (radial abduction), and FIG. 7d shows the end point of an ulnar deviation movement (ulnar abduction). The rotational movement around the deviation axis DA in one direction is terminated by the recurrence of an interaction between the corresponding edge of the second control slide 215 and the respective side edge 2123, here by way of example of the second side edge pair SP₂. The values of the swept-over angles |W_{D1}| (radial abduction) and |W_{D2}| (ulnar abduction) are the same in this embodiment. As the range of movement for the entire deviation movement urD permitted in this position of the second control slide 215, the sum of |W_{D1}| and |W_{D2}| is obtained, that is to say 2×|W_{D1}| here. By further guiding the second control slide 215 out of the first cutout 2122, the possible range of movement permitted by the control element 212 can be increased. It can be seen from FIGS. 7c and 7d that, when the second control slide 215 is removed slightly further from the rotary cylinder 2121 or the end edge 2128, at a rotation of the control element 212 about the deviation axis DA, the side edges 2123 of the second side edge pair SP₂ would no longer interact with the respective edges of the second control slide 215, but rather the side edges 2123 of the side edge pairs SP₃ or SP₄ arranged further toward the edge 2129 of the control element 212. The value of the total swept-over angle |W_{D1}|+|W_{D2}| would be correspondingly larger.

The designations of the individual side edge pairs SPₙ have been omitted for clarity in FIGS. 7a-d and 8a-d, but they can be gathered from FIG. 6b.

As an alternative to the embodiment of the orthosis joint 2 according to the invention shown in FIGS. 7a-7d, instead of one, two second control slides 215, which interact with the control element 212, for the control of the ulnar or radial deviation movement urD of the hand can also be provided, wherein the second control slides 215 are each of substantially cuboid-shaped configuration, and can have a length 2152, a width 2153 and a height 2154. In this embodiment, the sum of the widths 2153 preferably corresponds to the spacing d₁ of the opposite side edges 2123 of the first side edge pair SP₁ which is arranged closest to the rotary cylinder 2121. Each second control slide 215 can also be configured such that it can be inserted into the first cutout 2122 of the control element 212 and such that it can be removed from it, and can preferably interact with the side edges 2123.

FIG. 8 once again shows, in subfigures a to d, the interaction of two second control slides 215 with the control element 212 from FIG. 6a and b for illustration of the control of the deviation movement of the hand.

FIG. 8a corresponds to the situation shown in FIG. 7a: both second control slides 215 are inserted into the first cutout 2122 as far as the end edge 2128 of the first cutout 2122. Since the sum of the widths 2153 of the second control slides 215 corresponds to the spacing d₁ of the opposite side edges 2123 of the first side edge pair SP₁ which is arranged closest to the rotary cylinder 2121, both control slides 215 interact with the closest side edge 2123 to them of the first side edge pair SP₁ and a rotation of the control element 212 about the deviation axis DA running through the rotary cylinder 2121 is prevented. The embodiment shown here with two second control slides 215 now allows firstly the same control mechanism for a control of the ulnar and radial deviation movement urD of the hand as the embodiment shown in FIGS. 7a-d. The prerequisite for this is that the two second control slides 215 always assume the same positions within the first cutout 2122, that is, are always inserted into the first cutout 2122 or are removed from it to the same extent. For this control mechanism, reference is made to the description of FIGS. 7a-7d .

Secondly, the embodiment shown here with two second control slides 215, however, also allows an asymmetrical control of the deviation movement urD of the hand. In FIGS. 8b and 8c, a situation is illustrated by way of example, in which one of the second control slides 215 is removed a little in the direction of the edge 2129 of the control element 212 from the first cutout 2122, and the other second control slide 215 remains in its original position with contact with the end edge 2128 of the first cutout 2122. In the case of such a positioning of the two second control slides 215, the control element 212 can, by means of the rotary cylinder 2121, carry out only a rotation with respect to the deviation axis DA in the framework of a radial deviation (corresponding to a radial abduction), and a rotational movement via the starting position (FIG. 8b) in the direction of an ulnar deviation (corresponding to an ulnar abduction) is prevented by the interaction between an edge of the second control slide 215 which has remained in its original position and that side edge 2123 of the first side edge pair SP₁ which lies close to it. The value of the maximum possible radial deviation movement angle |W_{D1}| is, as shown and described in FIG. 7c for the configuration with a second control slide 215, determined by the interaction of an edge of the previously moved second control slide 215 with the side edge 2123 of the second side edge pair SP₂. If the value of the angle |W_{D1}| which can be swept over is greater, the second control slide 215 can be moved further away from the end edge 2128 of the first cutout 2122 and can then interact correspondingly with the side edge 2123 of the third SP₃ or fourth SP₄ side edge pair. Accordingly, of course, the upper second control slide 215 in FIGS. 8a-8d can remain in its starting position (see FIG. 8a) and the lower second control slide 215 (in FIGS. 8a-8d) can be moved out of the first cutout 2122, in order thus to block the radial deviation, but to allow the ulnar deviation (corresponding to an ulnar abduction). In FIG. 8d, a situation is finally shown, in which both second control slides 215 have been moved from their starting position in FIG. 8a in the direction of the edge 2129 of the control element 212 out of the first cutout 2122, wherein the movement within the first cutout 2122 of the two second control slides 215 is carried out to a different extent. The upper second control slide 215 in FIG. 8d is located in a position within the first cutout 2122, which allows an interaction with the side edges 2123 of the second side edge pair SP₂, whereas the lower second control slide 215 in FIG. 8d has been moved slightly further toward the edge 2129, with the result that an interaction with the side edges 2123 of the third side edge pair SP₃ is enabled. A rotation of the control element 212 by means of the rotary cylinder 2121 about the deviation axis DA in this positioning of the two second control slides 215 sweeps over a different angle range for the radial deviation (angular amount |W_{D1}|) and for the ulnar deviation (angular magnitude |W_{D2}|). In FIG. 8d, these two differently sized angles are labeled by means of dashed lines and curved double arrows. The total possible range of movement of the ulnar and radial deviation urD again results in the positioning shown here of the two second control slides 215 from the sum of the values |W_{D1}| and |W_{D2}|, wherein, in the present example, the permitted range of movement of the ulnar deviation (corresponding to |W_{D2}|) is greater than the permitted range of movement of the radial deviation (corresponding to |W_{D1}|). By different positioning of the two second control slides 215 within the first cutout 2122, a multiplicity of different ranges of movement of the ulnar and radial deviation urD can thus advantageously be controlled, which allows an individual adaptation to the respective health and/or rehabilitation state of the patient.

FIG. 9 shows one embodiment of an orthosis 1 according to the invention with an embodiment of an orthosis joint 2 according to the invention in a perspective view.

In this embodiment of the orthosis 1, the orthosis joint 2 is connected in particular via the base element 213 of the movement module 21 to a forearm fastening 11 of the orthosis 1. As an alternative, a portion of the forearm fastening 11 may also form the base element 213 of a movement module 21 and in particular a mount 2131 for the rotary cylinder 2121 of the control element 212 (not shown). The orthosis 1 or its forearm fastening 11 can also be reversibly fastened to the patient's forearm via fasteners 12. Hook and loop straps or belts with perforation and belt buckle in the widest sense are particularly suitable as fasteners 12. However, the fasteners 12 can also be prepared by a method of additive manufacturing (“3D printing”), for example as a belt-like continuation of the forearm fastening 11 and can be provided with a locking mechanism (e.g. by arrangement of nubs on one side of the band-like continuation and correspondingly formed holes on another band-like continuation of the forearm fastening 11), as is illustrated by way of example in FIG. 9. The forearm fastening 11 and the fastener or fasteners 12 can preferably be manufactured from a plastic.

In order to ensure an adjustment, which is safe, fast and can be carried out by the patient themselves without the use of tools, of predetermined deviation angles for a maximum possible ulnar and/or radial abduction (or ulnar/radial deviation movement urD), each second control slide 215 can also be configured to be reversibly fixed at predetermined positions during insertion into and removal from the first cutout 2122. For this purpose, a snap-in mechanism 2151 may preferably be provided. Each second control slide 215 may have, in particular on its side directed toward the orthosis 1, a sawtooth profile (not shown), which can reversibly enter into operative connection to a correspondingly designed side, facing the second control slide 215, of the orthosis 1 and/or of a housing 13 for the second control slide or slides 215 and can thus form a snap-in mechanism 2151. One embodiment of a housing 13 for the second control slide 215 is shown by way of example in FIGS. 1, 2 and 9. The second control slide 215 can also have an operating element 2155 for simple operation by the patient, by means of which it can be guided in particular within the housing 13 to the movement module 21 and thus to the first cutout 2122 of the control element 212 toward or away from the movement module 21 and thus away from the first cutout 2122.

Alternatively or cumulatively, the snap-in mechanism 2151 can also be formed by one or more, preferably spring-loaded, pins or nubs, which can be arranged on the sideof the orthosis 1 and/or of the housing 13, the control slide 215, directed toward the second control slide 215, and interact with correspondingly formed depressions, in particular holes, in the side, directed toward the orthosis 1, of the second control slide or slides 215. In FIGS. 1 and 2, such a snap-in mechanism 2151 is shown by way of example.

For the use of an orthosis 1 according to the invention by a patient, the pivot lever 22 which is operatively connected to the control element 212 of the movement module 21, as shown in FIG. 9, can particularly preferably be connected via a connecting means 3 to a hand fastening 4. The connecting means 3 can be connected fixedly to the hand fastening 4 or else can be of reversibly connectible configuration, for example, via a click-in or snap-in mechanism. With respect to the pivot lever 22, the connecting means 3 can preferably be mounted movably, as a result of which a change in the spacing between the movement module 21 and a contact point between the hand fastening 4 and the hand top side or hand can be advantageously taken into account during the execution of an extension and flexion movement E/F.

For the reversible connection of the hand of a patient to the hand fastening 4, finally a fastener 41 can be arranged at the hand fastening 4, wherein the fastener 41 can be formed in particular from a textile material, which is preferably designed so as to be connectible via a hook and loop connection to the bottom side of the hand fastening 4. The fastener 41 can advantageously enclose the thumb of the patient, for example by a hole within the textile material of the fastener 41, through which the thumb of the patient can be inserted and thus enclosed (see FIG. 9), or else by a fastener 41 which is formed from several textile bands, which can be placed around the patient's thumb accordingly (not shown). A fastener 41 of such configuration advantageously prevents the slipping of the orthosis 1 during movements of the patient.

FIG. 10 shows a further, alternative embodiment of an orthosis joint 2 according to the invention with two second control slides 215, wherein one of the control slides 215 is configured as a deviation control slide 218 and the other is configured as a fixing slide 219.

In this embodiment, therefore, preferably two second control slides 215—one configured as a deviation control slide 218 and one configured as a fixing slide 219—interact with the control element 212 for the control of the ulnar/radial deviation movement urD of the hand.

In FIGS. 11 and 12, embodiments of a deviation control slide 218 and a fixing slide 219 are each shown individually.

The second control slide 215, which is configured as a deviation control slide 218, can, as shown, preferably comprise a head region 2181 and a body region 2182, wherein the head region 2181 is configured to be inserted into the first cutout 2122 of the control element 212 and to be removed from it and to interact with the side edges 2123 of the first cutout 2122 of the control element 212. The body region 2182 of the deviation control slide 218 preferably comprises a guide track 2184 for a control pin 2169 of a second actuating means 2167, which is included by the orthosis joint 2. The second actuating means 2167 can be designed, for example, as a type of screw, which can be rotated clockwise and counter-clockwise via a groove 2168. The guide track 2184 may preferably be of approximately L-shaped configuration, wherein the transition from the longer part of the “L” to the shorter part of the “L” may preferably be of round configuration, and wherein the shorter part of the “L” can be oriented, in particular, toward the head region 2181 of the deviation control slide 218.

The head region 2181 of the deviation control slide 218 can be particularly preferably formed by two, mutually opposite lever elements 2183, which are resiliently movable with respect to each other. The spring action of the lever elements 2183 can be produced in particular firstly by the elastic properties, existing up to a certain degree, of the material used, from which the deviation lever 218 is manufactured. For this purpose, a metallic material, in particular aluminum, can be used, for example. Secondly, the lever elements 2183—alternatively and cumulatively—can also be designed structurally in such a way that a certain elasticity is produced. In FIG. 11, it is shown by way of example that the lever elements 2183 can be designed in particular as arms of a fork, which point away from the body region 2182 of the deviation control slide 218. The respective narrow configuration of the lever elements 2183, in particular at the transition to the body region 2182, advantageously produces a certain spring action of the respective lever elements 2183 when acted on from the outside here. In a relaxed state, that is to say without external influence on the lever elements 2183, the outer edges 2185 of said lever elements 2183 to each other can also preferably be at a spacing A from each other, which corresponds to the spacing d₁ of the opposite side edges 2123 of the first side edge pair SP₁, arranged closest to the rotary cylinder 2121, of the first cutout 2122.

The other second control slide 215 which is configured as a fixing slide 219 preferably likewise comprises at least one head region 2191 and one body region 2192, wherein the head region 2191 of the fixing slide 219 is configured in particular to interact with a fourth cutout 2120 of the control element 212. The orthosis joint 2 according to the invention preferably comprises in this embodiment at least one fourth cutout 2120, arranged on the control element 212, for deviation fixation. Preferably, however, a multiplicity of fourth cutouts 2120 are provided, as indicated by way of example in FIGS. 13b and 13c by four such fourth cutouts 2120.

The head region 2191 of the fixing slide 219 may comprise a head element 2193, which is fixedly connected to the body region 2192 of the fixing slide 219, for interaction with said fourth cutout 2120, as shown in FIG. 12. Away from the head element 2193, the head region 2191 of the fixing slide 219 can also be of concave configuration, with the result that, in an interaction of the head element 2193 with the fourth cutout 2120, the remaining head region 2191 of the fixing slide 219 can come into contact with the control element 212 (see FIG. 13c in this regard). Such a contact advantageously allows a further, more homogeneously distributed transmission of force from the control element 212 to the fixing slide 219, and thus advantageously strengthens the fixation of the deviation movement urD when the head element 2193 engages into the fourth cutout 2120.

The body region 2192 of the fixing slide 219 may in turn have a guide track 2194 for the control pin 2169 of the second actuating means 2167. Said guide track 2194 can, as can be seen in FIG. 12, preferably be of U-shaped configuration, wherein the opening of the “U” is preferably directed toward a longitudinal side of the fixing slide 219, and wherein that arm of the “U” which lies closer to the head region 2191 can preferably be shorter than that arm of the “U” which is further away from the head region 2191.

FIG. 13 shows now, in subfigures a to c, the interaction of the embodiment shown in FIG. 10 of an orthosis joint 2 with a deviation control slide 218 and a fixing slide 219 for illustration of the control and fixation of the deviation movement of the hand.

FIG. 13a shows, by way of example, the bottom side 2127 of the control element 212 with a deviation control slide 218 which is pushed in as far as the end edge 2128 of the first cutout 2122. If the control element 212 remains at rest with respect to the deviation slide 218, as shown here, the spacing A between the two lever elements 2183 preferably corresponds to the spacing d₁ between the two side edges 2123, which are closest to the rotary cylinder 2121, that is, the side edge pair SP₁ (see FIG. 13b). In this position of the deviation control slide 218, the deviation movement urD permitted by the orthosis joint 2 in both directions would be blocked apart from a very small angle range resulting from the above-mentioned spring action, by interactions of the outer edges 2185 of the deviation control slide 218 with the side edges 2123, and thus the hand would be fixed. A complete fixation can be ensured by a modification in particular of the base element 213 of the orthosis joint 2, which is described in greater detail below in connection with FIG. 14.

In FIG. 13a, it can also be seen that the deviation control slide 218 and the fixing slide 219 in particular can be movably mounted and can be configured, by means of the control pin 2169 of the second actuating means 2167, so as to be displaceable with respect to each other. The second actuating means 2167, which can be designed, for example, as a screw, which has, on one side, the control pin 2169 and, on an opposite side, a groove 2168, can interact by means of the control pin 2169 both with the deviation slide 218 and with the fixing slide 219. To this end, the control pin 2169 of the second actuating means 2167 can preferably be positioned such that it extends both into the guide track 2184 of the deviation control slide 218 and into the guide track 2194 of the fixing slide 219 and can move in both guide tracks 2184 and 2194.

In FIGS. 13a to c, the second actuating means 2167 (or its visible parts) and its control pin 2169 are shown in each case in a hatched manner. In FIGS. 13a and 13c, the two slides 218 and 219 are also “shown solidly”, with the result that the deviation control slide 218 covers the fixing slide 219 to varying degrees. In FIG. 13b, the deviation slide 218 is in contrast shown as an outline drawing, with the result that the fixing slide 219 shows through the deviation control slide 218 and the course of the two guide paths 2184 and 2194 of this embodiment of the invention is visible.

Due to the guide paths 2184 and 2194 which are shaped and oriented toward each other differently, in the case of a rotation of the second actuating means 2167, the deviation control slide 218 can initially be pushed away by the end edge 2128 of the first cutout 2122 in the direction of the fixing slide 219, wherein the fixing slide 219 can preferably initially remain in its position.

This can lead, for example, to an adjustment of the orthosis joint 2 as shown in FIG. 13b.

In this adjustment of the orthosis joint 2, the head region 2181 of the deviation control slide 218 extends to a certain degree into the first cutout 2122, wherein there is no longer any contact between the outer edges 2185 of the deviation control slide 218 and the side edges 2123 of the first cutout 2122. The control element 212 can rotate in this adjustment around the rotary cylinder 2121 or around its rotating axis DA extending through said rotary cylinder 2121, and the user can perform a deviation movement urD of the hand. In FIG. 13b, such a deviation movement urD is illustrated by way of example by a small curved arrow, which indicates a rotation of the control element 212 in the clockwise direction. However, the deviation movement urD is only possible in this adjustment until the head region 2181, in particular its lever element 2183, comes back into contact with a side edge 2123 of the first cutout 2122 (indicated by a straight dashed arrow in FIG. 13b). The mechanism functions here as in the other embodiments described further above (see FIGS. 7a to 7d and FIGS. 8a to 8d).

At the moment of contact between the lever element 2183 and the side edge 2123, said lever element 2183 advantageously dampens the stop due to its spring properties, with the result that the user can advantageously perceive stopping at the respective end points of the deviation movement urD as softer than in the two other embodiments of the present invention shown. In FIG. 13b, this possible damping movement at the top of the two lever elements 2183 is indicated by way of example by a small curved arrow.

FIG. 13c finally shows an adjustment of the orthosis joint 2, in which the second actuating means 2167 has been turned further until its control pin 2169 has moved almost to the end of the guide track 2184. During this rotation, the control pin 2169 pulls the deviation slide 218 firstly further out of the first cutout 2122, but secondly pushes the fixing slide 219 by about half of its travel advantageously in the direction of the first cutout 2122, with the result that finally an end position as shown in FIG. 13c can be reached.

The head element 2193 of the fixing slide 219 can advantageously engage in this end position into one of the fourth cutouts 2120, which are arranged on the control element 212, in particular within its first cutout 2122, and can in turn fix the orthosis joint 2 in this manner. If a multiplicity of fourth cutouts 2120 are provided, such as here for example four distributed over an angular range, the fixation can not only be carried out in a comparatively straight position of the hand, as shown here, but there can also advantageously be a fixation in the case of a hand which is angled away with respect to a deviation movement urD, depending on how the control element 212 has previously been rotated with respect to its axis of rotation DA.

FIG. 14 finally shows a view of one embodiment of a base element 213 of an orthosis joint 2 as shown in FIG. 10, and a deviation control slide 218 inserted therein.

As already mentioned above, due to the design of the head region 2181 of the deviation control slide 218, no complete fixation of the deviation movement urD can be achieved in this embodiment of the invention by simply pushing the deviation control slide 218 onto the end edge 2128 of the first cutout 2122, since the lever elements 2183 per se can always perform a certain spring movement. In order to also enable complete fixation in an extended hand position in this embodiment of the orthosis joint 2 according to the invention, as shown in FIG. 14, a stop pin 2133 can particularly preferably be arranged on the base element 213 of the orthosis joint 2. This stop pin 2133 is preferably positioned on the base element 213 and firmly connected to this in such a way that said stop pin 2133 is located, in the case of a deviation control slide 218 completely pushed toward the end edge 2128 (i.e. the situation shown in FIG. 13a), exactly between the lever elements 2183. The stop pin 2133 then advantageously prevents a spring movement of the lever arms 2183 inward, i.e. toward each other, when force is applied to the outer edges 2185 of the deviation slide 218 via the respective side edges 2123 of the first side edge pair SP₁ and thus also advantageously enables a complete fixation of the hand in this embodiment.

The present invention relates to an adjustable orthosis joint 2 for controlled movement and/or fixation of a hand and to an orthosis 1 with such an orthosis joint 2. The orthosis joint 2 according to the invention is characterized by a movement module 21, which comprises at least one base element 213 and at least one control element 212 with a top side 2126 and a bottom side 2127 which faces the base element 213 and lies opposite the top side 2126, wherein a rotary cylinder 2121 is arranged on the bottom side 2127, by means of which rotary cylinder the control element 212 is rotatably mounted relative to the base element 213 with respect to a deviation axis DA, wherein the control element 212 is configured to be limited in a controlled manner in its rotational movement, and wherein the control element 212 is configured to limit the movement of a pivot lever 22, which is operatively connected to the movement module 21, in a controlled manner in its range of movement. It can be adjusted by the patient themselves without tools, and is of such compact construction that its use in everyday life is not perceived as disruptive.

### LIST OF DESIGNATIONS

1 orthosis
11 forearm fastening
12 fastener
13 housing for second control slide (215)
2 orthosis joint
21 movement module
211 cover element
2111 cutout for pivoting lever (22)
212 control element
2121 rotary cylinder
2122 first cutout for deviation control
2123 side edge of the cutout (2122)
2124 second cutout for pivoting lever (22)
2125 third cutout for first control slide (214)
2126 top side
2127 bottom side
2128 end edge of the cutout (2122)
2129 edge
2120 fourth cutout for deviation fixation
213 base element
2131 mount for rotary cylinder (2121)
2132 guide means for rotary cylinder (2121)
2133 stop pin
214 first control slide for extension/flexion (E/F)
2141 cut-out for first actuating means (216)
2142 fork-shaped end
2143 connecting pin
215 second control slide for ulnar/radial deviation (urD) of the hand
2151 snap-in mechanism
2152 length
2153 width
2154 height
2155 operating means
216 first actuating means
2161 head
2162 groove
2163 collar
2164 actuating cylinder
2165 longitudinal axis
2166 channel
2167 second actuating means
2168 groove
2169 control pin
217 snap-in mechanism
2171 spring
2172 depression
218 deviation control slide
2181 head region
2182 body region
2183 lever element of the deviation control slide (218)
2184 guide track of the deviation control slide (218)
2185 outer edge of the lever element (2183)
219 fixing slide
2191 head region of the fixing slide (219)
2192 body region of the fixing slide (219)
2193 head element of the fixing slide (219)
2194 guide track of the fixing slide (219)
22 pivot lever for extension/flexion (E/F)
221 pivot axis
3 connecting means
4 hand fastening
41 fastener
A spacing of the outer edges (2185) of the lever elements (2183)
LA longitudinal axis of the orthosis (1)
DA deviation axis of the orthosis joint (2)
E/F extension and flexion of the hand
urD ulnar and radial deviation of the hand
dₙ spacing of the cutout edges (2123)
H height of the control element (212) in the region of the first cutout (2122)
H height of the control element (212) outside the region of the cutout (2122)
SPₙ side edge pair
   |W_{D1}|, |W_{D2}| value of the deviation angle
   |W_{E/F}| value of the extension/flexion angle

1. An adjustable orthosis joint (2) for the controlled movement and/or fixation of a patient's hand, at least comprising
a movement module (21) for controlling an extension and/or flexion movement (E/F) and for controlling and executing an ulnar and/or radial deviation (urD) of the hand; and
a pivot lever (22), which is operatively connected to the movement module (21), for carrying out the extension and/or flexion movement (E/F) of the hand;
characterized in that
the movement module (21) comprises
at least one base element (213) and
at least one control element (212) with a top side (2126) and a bottom side (2127) facing the base element (213) and lying opposite the top side (2126),
wherein a rotary cylinder (2121), by way of which the control element (212) is mounted rotatably relative to the base element (213) with respect to a deviation axis (DA), is arranged on the bottom side (2127),
wherein the control element (212) is designed such that its rotational movement around the deviation axis (DA) can be restricted in a controlled manner,
and wherein the control element (212) is designed to restrict the range of movement of the pivot lever (22) in a controlled manner.

2. The orthosis joint (2) as claimed in claim 1, characterized in that the control element (212) comprises at least one first control slide (214), which can be moved by means of at least one first actuating means (216) with respect to the pivot lever (22) which is operatively connected to the movement module (21), wherein the first control slide (214) is preferably of substantially cuboid-shaped configuration and, at its end facing the pivot lever (22), is of fork-shaped configuration.

3. The orthosis joint (2) as claimed in claim 2, characterized in that the first actuating means (216) comprises at least one head (2161) and one actuating cylinder (2164) which are connected to each other, preferably via a collar (2163), with the result that a component with a longitudinal axis (2165) is formed,
wherein the actuating cylinder (2164) has a circular cross section and is connected to the head (2161) of the first actuating means (216) eccentrically with respect to the longitudinal axis (2165).

4. The orthosis joint (2) as claimed in claim 3, characterized in that the first control slide (214) comprises at least one cutout (2141) which is configured to receive the actuating cylinder (2164) and to interact with it.

5. The orthosis joint (2) as claimed in claim 2, characterized in that the first actuating means (216) comprises at least one head (2161) and one actuating cylinder (2164) which are connected to each other, preferably via a collar (2163), with the result that a component with a longitudinal axis (2165) is formed,
wherein the actuating cylinder (2164), on its side facing away from the head (2161), has a preferably substantially C-shaped channel (2166).

6. The orthosis joint (2) as claimed in claim 5, characterized in that the first control slide (214) comprises a connecting pin (2143) which engages in the channel (2166) of the actuating cylinder (2164) during use of the orthosis joint (2),
wherein the connecting pin (2143) is preferably arranged within a cutout (2141) of the first control slide (214), which is configured to receive the actuating cylinder (2164).

7. The orthosis joint (2) as claimed in one or more of claims 2 to 6, characterized in that the first actuating means (216) and/or the first control slide (214) comprises at least one snap-in mechanism (217) which is configured to reversibly fix the first control slide (214) in its displacement movement with respect to the pivot lever (22) which is operatively connected to the movement module (21) at at least one predetermined position.

8. The orthosis joint (2) as claimed in one or more of the preceding claims, characterized in that the control element (212) comprises a first cutout (2122) for deviation control,
which is arranged on the bottom side (2127) of the control element (212);
and which extends from an edge (2129) of the control element (212) in the direction of the rotary cylinder (2121), with the result that a height (h) of the control element (212) in the region of the first cutout (2122) is smaller than a height (H) of the control element (212) in the region outside the first cutout (2122),
as a result of which at least four side edges (2123) are formed which delimit the region within the first cutout (2122) from the area outside the first cutout (2122),
of which two lie substantially opposite each other at a spacing dₙ where n=1, 2, . . . and form a side edge pair SPₙ where n=1, 2, . . . ;
wherein a spacing dₙ between two side edges (2123), which are arranged closer to the rotary cylinder (2121) and lie opposite one another, of a side edge pair SPₙ is always smaller than a spacing dₙ₊₁ between two side edges (2123), which are arranged closer to the edge (2129) of the control element (212) and lie opposite one another, of a side edge pair SPₙ₊₁.

9. The orthosis joint (2) as claimed in claim 8, characterized in that a multiplicity of side edges (2123), preferably eight side edges (2123), are formed, of which two each lie substantially opposite each other and form a side edge pair SPₙ where n=1,2, . . . .

10. The orthosis joint (2) as claimed in claim 8 or 9, characterized by a second control slide (215), which interacts with the control element (212), for the control of the ulnar/radial deviation movement (urD) of the hand,
wherein the second control slide (215) is of substantially cuboid-shaped configuration and has a length (2152), a width (2153) and a height (2154);
wherein the width (2153) corresponds to the spacing d₁ of the opposite side edges (2123) of the first side edge pair SP₁ arranged closest to the rotary cylinder (2121);
and wherein the second control slide (215) is configured such that it can be inserted into the first cutout (2122) of the control element (212) and can be removed from it and can interact with the side edges (2123).

11. The orthosis joint (2) as claimed in claim 8 or 9, characterized by two second control slides (215) which interact with the control element (212) for the control of the ulnar/radial deviation movement (urD) of the hand,
wherein the second control slides (215) are each of substantially cuboid-shaped configuration and have a length (2152), a width (2153) and a height (2154);
wherein the sum of the widths (2153) corresponds to the spacing d₁ of the opposite side edges (2123) of the first pair of side edges SP₁ arranged closest to the rotary cylinder (2121);
and wherein each second control slide (215) is configured such that it can be inserted into the first cutout (2122) of the control element (212) and can be removed from it and can interact with the side edges (2123).

12. The orthosis joint (2) as claimed in claim 10 or 11, characterized in that each second control slide (215) is configured to be reversibly fixed at predetermined positions during insertion into and removal from the first cutout (2122), preferably by means of a snap-in mechanism (2151).

13. The orthosis joint (2) as claimed in claim 8 or 9, characterized by two second control slides (215) which interact with the control element (212) for the control of the ulnar/radial deviation movement (urD) of the hand and at least one fourth cutout (2120) which is arranged on the control element (212) for the deviation fixation;
wherein one of the second control slides (215) is designed as a deviation control slide (218), at least comprising:
a head region (2181) which is configured to be inserted into the first cutout (2122) of the control element (212) and to be removed from it, and to interact with the side edges (2123) of the first cutout (2122) of the control element (212),
and a body region (2182) which has a guide track (2184) for a control pin (2169) of a second actuating means (2167) which is covered by the orthosis joint (2);
wherein the other of the two second control slides (215) is designed as a fixing slide (219), at least comprising:
a head region (2191) which is configured to interact with the fourth cutout (2120) of the control element (212),
and a body region (2192) which has a guide track (2194) for the control pin (2169) of the second actuating means (2167);
and wherein the deviation control slide (218) and the fixing slide (219) are displaceably mounted and configured by means of the control pin (2169) of the second actuating means (2167) in a displaceable manner with respect to each other.

14. The orthosis joint (2) as claimed in claim 13, characterized
in that the head region (2181) of the deviation control slide (218) is formed by two opposite lever elements (2183) which are resiliently movable with respect to each other and the outer edges (2185) of which are at a spacing (A) from each other in a relaxed state, which spacing corresponds to the spacing d₁ of the opposite side edges (2123) of the first side edge pair SP₁, arranged closest to the rotary cylinder (2121), of the first cutout (2122);
and in that the head region (2191) of the fixing slide (219) comprises a head element (2193) for interaction with the fourth cutout (2120), which is firmly connected to the body region (2192) of the fixing slide (219).

15. An orthosis (1) for the controlled movement and/or fixation of a hand or a hand and a forearm of a patient, characterized by an adjustable orthosis joint (2) as claimed in one or more of the preceding claims 1 to 14.
